(19) United States
(12) Patent Application Publication
(10) Pub. No.: US 2012/0136327 A1
(43) Pub. Date: May 31, 2012

(54) Personalized and Concealable Sanitary Absorbent Article

(76) Inventor: Susan Jones, (US)

(21) Appl. No.: 13/305,681

(22) Filed: Nov. 28, 2011

Related U.S. Application Data
(60) Provisional application No. 61/417,693, filed on Nov. 29, 2010.

(51) Int. Cl. A61L 15/46; A61F 13/60

(52) U.S. Cl. 604/359; 604/385.05

(57) ABSTRACT

Disclosed is a colored absorbent article, comprising a sanitary pad, panty shield and liner that is adapted to provide a user a means to conceal the presence of the article by matching its outward appearance with the user's undergarments, while providing a means to easily monitor menstruation from the interior surface of the article. The article comprises an inner layer, one or a plurality of outer layers and a peelable film, revealing adhesive on the outer layer surface. In a preferred embodiment, the pad comprises an inner absorbent layer of durable, breathable and absorbent material, backed by an outer layer that is color matched with an undergarment and having a removable film outer surface with an uplifting message thereon. Alternate embodiments contemplate several outer layers, each layer having a different color to match a different undergarment. The article is biodegradable and comprises an imbedded fragrance that matches its outward coloration.

### CROSS REFERENCE TO RELATED APPLICATION

This application claims the benefit of U.S. Provisional Application No. 61/417,693 filed on Nov. 29, 2010, entitled “Suzy's Pretty Panty Liners and Pads.”

### BACKGROUND OF THE INVENTION

1. Field of the Invention

The present invention relates to female absorbent articles, such as sanitary pads and liners for the purposes of absorbing vaginal, menstrual or incontinence discharge. More specifically, the present invention pertains to an improved absorbent article wherein the outward appearance of the device is adapted to match an undergarment and conceal its presence, while the inward surface provides visualization of discharge, allowing the user to monitor her body and menstrual conditions during heavy or lighter period flow.

2. Description of the Prior Art

Absorbent articles, such as female sanitary pads and liners of the present invention, are devices utilized to retain and capture vaginal fluid from the crotch region of a female during menstruation or in the case of incontinence. Their structure employs common elements for absorption of fluid and attachment to an undergarment during use. Absorption is generally performed by an inner core of absorbent material, wherein fluid is captured and retained therein, preventing fluid from spreading onto the user or her clothes. This prevents discomfort and embarrassment during menstruation or for those with incontinence, wherein the pad prevents the outward appearance of any leaking fluid. Common means of attachment to undergarment articles are traditionally adhesive outer surfaces or tabs that fold around the periphery of an undergarment crotch region to maintain the article position while permitting the user to engage in normal activity.

Traditional pads and panty liners are also white cotton or a similar material for the purpose of absorption and comfort against the user's skin. While and effective means of trapping fluid, these articles provide little in the way of concealment against colored undergarments, wherein the white pad is clearly visible from the exterior of the undergarment. In these instances, women are left with little choice but to wear white undergarments or allow the sanitary pad to be seen through the undergarment. Attempts have been made to provide colored panties; however these devices lack the versatility of the present invention and fail to provide a plurality of colors in a single pad or multiple colors in a package of sanitary pads.

Similar drawbacks to the currently available sanitary articles are their inherent construction, wherein they lack biodegradable material that would allow their structure to break down easily in the environment and provide a lower footprint in landfills. The present invention provides a pad made entirely of a biodegradable fabric material that is capable of functioning as an absorbent article, but also breaks down after a shorter time period. Use of plastics or synthetic material that does not easily biodegrade is not utilized in the construction of the present invention, despite their clear utility in the structure of current sanitary absorption articles. Further still, current sanitary pads provide little in the way of fragrance to mask odor while in use. The present invention provides a scented or unscented article that matches its exterior color, providing the user with a novel odor mask that is aligned with its outward appearance and that of the undergarment to suit the mood and preference of a user.

Devices have been disclosed in the prior art that describe female sanitary pads with inherent color and masking qualities. These devices have familiar design features for providing a female absorbent article, and further disclose colored pads and liners. However, these devices fail to disclose the structure of the present invention and the elements that comprises its masking features. Little is provided in the way of biodegradability or consideration for the quantities of pads that may be produced and ultimately end in a landfill site. The present invention provides a means to combat these known problems and drawbacks in the art.

U.S. Published Patent Application Publication No. 2003/0109839 to Costea is one such device in the art that discloses a dark colored absorbent article, such as a panty liner or sanitary napkin, comprising an apertured top sheet, and underlying layer visible through the apertures of the top sheet and back sheet. The top sheet and underlying layer have an L Hunter color value of less than 60 to provide optimum masking and discretion of the pad while in use in conjunction with a female undergarment. Blood and menstrual fluid are therefore not easily visualized through the pad, as the color of the pad is ideally black or having an L Hunter value of less than 60. While providing a colored sanitary pad, the Costea device construction differs from the present invention, wherein a plurality of colors is possible from a single pad.

U.S. Published Patent Application Publication No. 2003/0208177 to D'Alessio is another device that describes a menstrual absorbent article designed to be worn in or attached to thong slips or G-string undergarments. The article follows the general narrow contour of these undergarments and attaches thereto. The device comprises an absorbent core, side flaps and a top sheet side with an article coloration to align the pad with fashion aspects of currently frequented thong undergarments. While providing a colored pad to match undergarments, the D'Alessio disclosure does not disclose an undergarment having a plurality of color options or the construction of the present invention.

Finally, U.S. Pat. No. 6,932,798 to Kudo discloses a colored absorbent article adapted to attach to the inner side of an undergarment and matching in color with that article. The device comprises a body surface facing the skin of a wearer and an outer garment surface facing the inner side of the garment. The body surface is provided in a color range suitable for visualization of menstrual blood and discharge. Examples and evaluations of an acceptable color is provided for providing excellent body surface visualization, while the outer surface is adapted to be provided in a specific color to match an undergarment. The present invention provides a similar pad but constructed of a plurality of available outer colors in one pad, and further in a biodegradable form with an imbedded scent to match the provided color. The interior, user-facing surface of the present pad is adapted to be a lighter, white color for visualization and monitoring of the menstruation of the user wearing the device. The outward appearance is adapted to be chosen for a particular undergarment for matching its color and concealing the presence of the pad while in use.

Further, U.S. Published Patent Application Publication No. 2006/0025735 to Berg discloses a disposable absorbent article having a back sheet, top sheet and an absorbent element. The article comprises an outer coloring that prevents its visibility and recognition when in use. Similar to the aforementioned disclosures, the Berg devices fails to disclose the elements of the present invention, wherein biodegradable, scent-matched article is provided with a colored backing, and alternatively one that provides a plurality of revealable colors thereon to match different colored undergarments without searching for multiple articles to match the color accordingly.

The present invention advances the art of colored sanitary articles by providing several improvements thereto, which allow the device to be concealed during use and prevent its unwanted outward visibility when utilized with a colored undergarment. The device provides a biodegradable structure that is functional as an absorbent pad, but is able to biodegrade after a defined period and after use. The outward color is provided with a matching scent, while the colored outer surface may employ a plurality of layers to allow several color choices prior to application thereof. The prior art provides various absorbent articles with a defined color palette for specific requirements, but fail to disclose the structure and spirit of the instant invention. It is therefore submitted that the present invention substantially diverges in design elements from the prior art, and consequently it is clear that there is a need for improvement to existing colored sanitary pad devices. In this regard the instant invention substantially fulfills these needs.

### SUMMARY OF THE INVENTION

In view of the foregoing disadvantages inherent in the known types of colored sanitary pads now present in the prior art, the present invention provides a new sanitary pad wherein the same can be utilized for providing convenience for the user when concealing the placement of a sanitary pad on a colored undergarment, masking any odor and providing an environmentally conscious product.

It is therefore an object of the present invention to provide a new and improved sanitary absorbent article that has all of the advantages of the prior art and none of the disadvantages.

Another object of the present invention to provide a female sanitary pad that comprises an inner absorbing core for capturing fluid and one that is attachable to an undergarment utilizing an adhesive outer surface.

Another object of the present invention is to provide a female sanitary pad having a colored outer surface and a fragrant scent to match the outer color, allowing concealment of the pad when utilized with colored undergarments and masking of unwanted odor.

Still another object of the present invention is to provide a female sanitary pad having a removable film covering its adhesive, colored outer layer, and further providing a plurality of removable outer films that reveal several color choices.

Yet another object of the present invention is to provide a female sanitary pad having a biodegradable construction to provide an environmentally conscious, disposable article.

A final object of the present invention is to provide a female sanitary pad having an outer, removable film covering an adhesive surface, wherein the film comprises an uplifting or inspiring message thereon for users to read when applying the pad. The pad comprises a modern product for the modern woman, wherein the construction of the article provides concealed application and the imbedded message provides a smile-evoking message, quote or anecdote for comforting or soothing a woman during times of physical discomfort.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the preferred embodiment of the sanitary pad, wherein the outer adhesive film is peeled back to reveal the outer surface coloration, along with a close-up cross section view thereof.

FIG. 2 shows a perspective view of an alternate embodiment of the sanitary pad, wherein an outer adhesive film is peeled back to reveal the outer surface coloration and a plurality of further removable films and color options, along with a close-up cross section view thereof.

FIG. 3 shows a perspective view of the preferred embodiment of the sanitary pad, wherein the outer adhesive film peeled back to reveal an internal, uplifting message on the film interior surface.

### DETAILED DESCRIPTION OF THE INVENTION

Reference is made herein to the attached drawings. Like reference numerals are used throughout the drawings to depict like or similar elements of the absorbent article. For the purposes of presenting a brief and clear description of the present invention, the preferred embodiment will be discussed as used for having a colored outer surface to conceal its presence. The figures are intended for representative purposes only and should not be considered to be limiting in any respect.

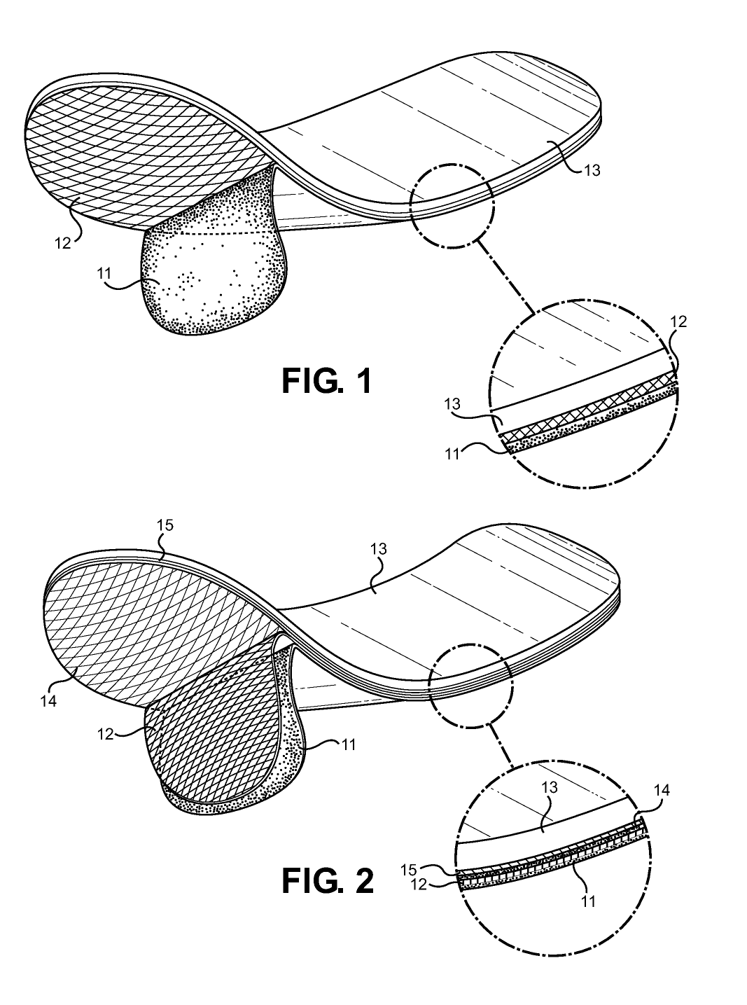

Referring now to FIG. 1, there is shown a perspective view of the preferred embodiment of the sanitary absorbent article, wherein the pad comprises an inner layer 13 having an absorbent material or absorbent inner core, a colored outer layer 12 and a removable film 11 that reveals an adhesive outer surface. The pad is designed to provide an absorbent sanitary napkin, pad or panty liner that can retain discharge, blood and other fluids while preventing leaking thereof and moisture buildup. The pad inner layer 13 is adapted for placement against the skin of a user, wherein fluid is trapped within the inner layer 13 core and prevented from leaking onto the user's undergarment. This layer is made of a biodegradable absorbent material adapted for trapping liquid. The outer layer 12 provides a colored outer surface to match the undergarment of the user and conceal its presence while in use. A peelable, removable film 11 covers the adhesive outer surface, wherein the film 11 is removed prior to application onto a user's undergarments. The outer surface is adapted to be pressed against the crotch region of an undergarment, wherein the adhesive secures the pad in position and prevents dislodgement thereof, permitting normal activity of the user.

The materials of the pad are adapted to be provided in a biodegradable form, wherein after the outer film 11 is removed, the pad is utilized and subsequently disposed of, it is easily biodegradable in a landfill setting. The goal is to provide not only a highly effective absorbent pad, but also to provide one that is environmentally conscious. This is growing concern in our society, as waste products are damaging our environment, and the proliferation of plastics and synthetic material has led to waste that does not break down naturally or does so over an unacceptably long period that is ultimately unsustainable with regard to the world population growth. When utilized, the user chooses a pad based on its outer surface coloration, which is preferably matched to the user's undergarment. The film 11 is removed from the outer layer 12 surface and subsequently disposed of. This film 11 is adapted to be a water soluble, fibrous or similar degradable material. Its purpose is not to absorb moisture, but rather act as a temporary adhesive cover. Its structure is designed for placement into a waste bin or alternatively a toilet after its removal. Example materials that are contemplated for its construction include polyvinyl alcohol (PVA) or a bioplastic material derived from biomass sources, such as starches, vegetable fats and oils. The inner and outer layer of the pad is preferably an organic cotton material, which is highly absorbent but breaks down naturally in the environment. Materials of similar quality, which provide an adequate level of moisture absorption while remaining biodegradable, are contemplated for use in the construction of the present invention.

The outer layer surface is pigmented or similarly colored in a particular color and shade to allow the user to choose a pad that closely resembles the color of her undergarments. This allows the pad to blend into the color of the undergarment, and be less conspicuous when placed thereon. Traditional white sanitary pads are clearly visible through colored undergarments, and therefore afford very little concealment for those wishing discreet application thereof.

Referring now to FIG. 2, there is shown a perspective view of an alternate embodiment of the sanitary pad of the present invention. In this embodiment, a plurality of outer layers 12, 14, and 15 and provided, each with a different outer surface color. The outer film 11 is removed from the outer most layer 12. If this color does not match the user's undergarment, the outer most layer 12 is removed, revealing a second most outer layer 14 with an adhesive outer surface and a second outer color. It is desired to disclose a construction of a colored pad that provides several layers of different color in this manner to permit a single pad to accommodate a random color, and preventing the user from storing several different colored pads and utilizing the one that closely matches her undergarment. In this way, a single pad can take the place of several different colored pads, and the plurality of layers each contain a differing outer surface color with adhesive thereon. The unused outer layers not desired are discarded prior to installation onto the crotch of the undergarment. Similar to the preferred embodiment, the waste from the removed layers, along with the construction of the pad are all biodegradable, preventing dangerous waste products and any worry discarding unused layers.

The colors of the disclosed pad may take any color, including primary colors, black or any shade/hue thereof to provide a means to conceal the pad from the exterior of a user's undergarment. Additionally, it is desired that the pad outer layer provide a scent, which may be a single scent for any color, or coordinating the color with a particular scent (i.e. red smells like cherry or strawberry, blue smells of fresh ocean breeze, etc.). The scent is particularly useful, as unwanted odors are masked and the pad provides a fresh smelling fragrance that may accompany the color of the undergarment and suits a woman's mood and boost her self confidence.

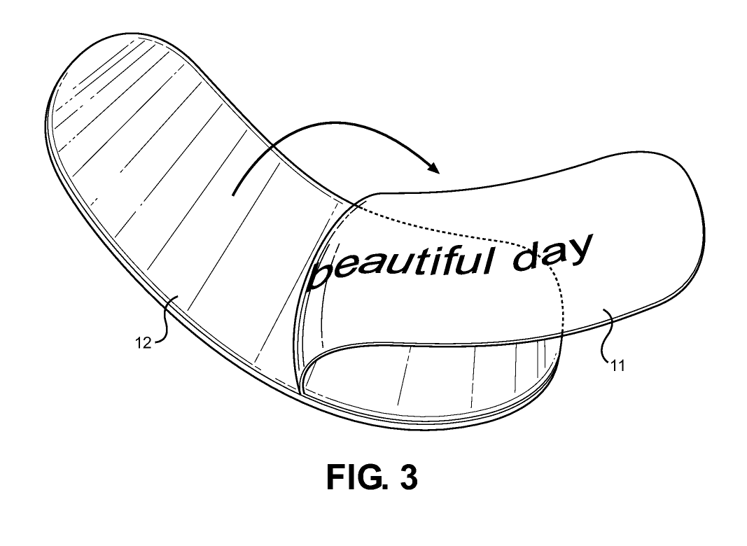

Referring now to FIG. 3, there is shown perspective view of the present invention, wherein the outer layer film 11 is being completely peeled from the colored outer layer 12. The interior surface of the film 11, which is adapted to mate against the outer layer 12 prior to being peeled, is adorned with an uplifting, motivational or inspiring message thereon for the user to read when applying the pad and removing the film 11. This message may be a quote, fact or anecdote that is adapted to inspire or uplift the user. The message is not visible from the exterior side of the film 11, and is only accessible when peeled from the outer layer.

Feminine hygiene products are necessary articles for those times when females are menstruating, when extra protection under their clothing is desired or for those with incontinence. However, traditional feminine hygiene products are often made in white or very light colors, making them highly visible through undergarment fabric. This is unsightly, and can make women feel embarrassed and self-conscious during their use. Women may resort to wearing less attractive underwear styles due to lack of confidence. The goal of the present invention is to disclose a discreet and easily concealable alternative to traditional feminine hygiene products. The device comprises a multi-layered absorbent pad that can be customized to color-coordinate with different pairs of underwear. The pads and liners are adapted to conform to the contours of a woman's body and adhere to the interior crotch region of the undergarment. They are further adapted to remain within the confines of different styles of underwear.

The construction of the pad is one that reduces the environmental impact of disposable sanitary pads, as the disclosed pad is designed to be constructed of natural or easily biodegradable materials, while still fulfilling its intended goal of concealed fluid absorption. The alternate embodiment of the disclosed pad further allows a single pad to be utilized on a variety of different color undergarments, wherein the outer layers are removable to reveal different outer layer colors. Further, the removable film that protects the adhesive layer prior to its use contains an inspirational or uplifting phrase on its interior surface, revealable upon peeling the film and exposing the adhesive of the outer most layer.

The present invention is intended to perform as a sanitary, absorbent article for feminine hygiene, wherein its structure may take several forms to conform to a plurality of different style undergarments or functions. It is desired to disclose the elements of the present invention for use as a panty shield, a sanitary pad or as a panty liner having either winged or curved construction. The winged construction of the sanitary pad allows for extended coverage and stability, as the surface area of the pad is expanded and the wings are able to wrap around the crotch region of an undergarment. Curved construction allows for the pad use with thinner-style undergarments, such as thongs and G-strings. It is not desired to limit the style of the absorbent article to a particular construction, but rather it is desired to disclose a biodegradable, color-matching device with an imbedded, uplifting message and an available scent to suit the user's preferences or mood.

Overall, the present invention provides a discreet device that allows a user to wear a feminine hygiene product in conjunction with stylish, revealing undergarments or those of a particular color. Unlike traditional feminine products, which are traditionally only available in lighter colors, the disclosed invention does not show through a woman's undergarment, making them safe for use with sheer and lace-style undergarments. The design of the layers, their construction and the interior message applied to the film provide a user with a unique sanitary pad that does not limit their undergarments to a specific style or color, and further reduces the harmful effects of a disposable pad on the environment.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) An absorbent article, comprising:
an absorbent inner layer and an outer layer having an adhesive outer surface;
a removable film covering said adhesive outer surface;
said adhesive adapted to attach to an undergarment of user, said outer surface having a coloration to match said undergarment;
said inner layer, outer layer and film constructed of a biodegradable material.

2) The device of claim 1, wherein said film further comprises:
an interior and exterior surface, said interior surface adapted to mate with said outer layer adhesive outer surface prior to attachment to an undergarment;
said film interior surface having a message thereon, revealable when said film is separated from said adhesive.

3) The device of claim 1, wherein said outer layer having an imbedded scent to mask odor.

4) The device of claim 3, wherein said scent coordinates with said outer surface.

5) An absorbent article, comprising:
an absorbent inner layer and a plurality of stacked outer layers, each having adhesive outer surfaces;
a removable film covering said outer most adhesive surface;
said adhesive outer surfaces adapted to join each of said plurality of outer surfaces, said outer most adhesive surface adapted to attach to an undergarment of user,
said outer layers each having a different coloration to match an undergarment;
said inner layer, outer layer and film constructed of a biodegradable material.

6) The device of claim 5, wherein said film further comprises:
an interior and exterior surface, said interior surface adapted to mate with said outer layer adhesive outer surface prior to attachment to an undergarment;
said film interior surface having a message thereon, revealable when said film is separated from said adhesive.

7) The device of claim 5, wherein said outer layer having an imbedded scent to mask odor.

8) The device of claim 7, wherein said scent coordinates with said outer surface.
